(19) United States
(12) Patent Application Publication
(10) Pub. No.: US 2016/0089110 A1
(43) Pub. Date: Mar. 31, 2016

(54) Conformal interface for medical diagnostic ultrasound volume imaging

(71) Applicant: Siemens Medical Solutions USA, Inc., Malvern, PA (US)

(72) Inventors: Andrzej Milkowski, Issaquah, WA (US); Richard Barr, Campbell, OH (US); Stephen R. Barnes, Bellevue, WA (US); Thomas Clary, Sammamish, WA (US)

(21) Appl. No.: 14/500,833

(22) Filed: Sep. 29, 2014

(51) Int. Cl. A61B 8/00; A61B 19/00; A61B 8/08

(52) U.S. Cl. CPC A61B 8/4281; A61B 8/429; A61B 8/483; A61B 8/4483; A61B 8/0825; A61B 8/5207; A61B 19/2203

(57) ABSTRACT

An ultrasound imaging standoff has a stiffer portion for contact with a volume transducer and a flexible portion filled with viscous fluid for conforming to the patient, resulting in fewer or no air gaps. Pressure is applied through the standoff to cause the connective tissue to flatten, resulting in less shadowing artifact.

### BACKGROUND

Volumetric ultrasound is used to image a patient, such as imaging women with dense breasts. There are a number of drawbacks with volume imaging dense breasts. The volume scanning transducer may not make contact with a large fraction, or in the case of a smaller breast, the entire surface of the breast. This results in air or other gaps between parts of the transducer and the patient, limiting the volume that can be scanned from a given position. The sonographer then repeats volume scans from other positions, requiring registration of separately acquired volumes. Another issue in volume ultrasound is shadowing. The nipple and other connective breast tissue cause acoustic shadows. The acoustic shadows cause image artifacts.

### BRIEF SUMMARY

By way of introduction, the preferred embodiments described below include methods, standoffs, conformal interfaces, transducer arrays, and systems for ultrasound imaging. A standoff has a stiffer portion for contact with a volume transducer and a flexible portion filled with viscous fluid for conforming to the patient, resulting in fewer or no air gaps. Pressure is applied through the standoff to cause the connective tissue to flatten, resulting in less shadowing artifact.

In a first aspect, a conformal interface is provided for ultrasound imaging. A sealed container has a first surface stiffer than an opposing second surface. The second surface is conformal to an outside surface of a patient, and the first surface is configured for acoustic contact with an ultrasound transducer array. A fluid more viscous than water in the sealed container.

In a second aspect, a method is provided for scanning a breast in medical diagnostic ultrasound volume imaging. A bag filled with a viscous fluid under pressure is placed against the breast of the patient. A volume scanning transducer is positioned against the bag. Pressure is applied with the volume scanning transducer to the bag. The pressure is greater than caused by gravity. The breast is volume scanned through the bag with the volume scanning transducer.

In a third aspect, a standoff is provided for ultrasound imaging. A housing contains a viscous fluid. The housing is, in a first part, a flexible, elastic material and is, in a second part, a relatively more stiff material. The viscous fluid and the housing being acoustically conductive.

The present embodiments are defined by the following claims, and nothing in this section should be taken as a limitation on those claims. Any one or combinations of any two or more of the aspects discussed above may be used. Further aspects and advantages of the invention are discussed below in conjunction with the preferred embodiments.

### BRIEF DESCRIPTION OF SEVERAL VIEWS OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 illustrates one embodiment of a conformal interface for ultrasound scanning;

FIG. 2 is a flow chart diagram of one embodiment of a method for ultrasound imaging with a standoff;

FIG. 3 illustrates an example interaction of the conformal interface of FIG. 1 with a breast; and

FIG. 4 is a block diagram of one embodiment of an ultrasound system for volume scanning.

### DETAILED DESCRIPTION OF THE PREFERRED EMBODIMENTS

A conformal, shadow-reducing interface is used for ultrasound volume or other scanning. The conformal interface is a sealed viscous, acoustically-conductive, fluid-filled bag. The bag creates a conformal interface between a transducer and the breast of the patient. Every breast has a different shape and structure. The bag may create a high-pressure interface between a chestwardly compressed transducer and the breast of a supine patient. A conformal design increases acoustic contact, patient comfort, and results in shadow reduction.

A volumetric ultrasound system may capture an entire, smaller dense breast in one volume acquisition with shadowing similar to traditional hand-held ultrasound. The conformal interface may provide for acoustic paths without gaps to a greater portion of the breast.

Breast imaging is used in the examples herein. In other embodiments, the conformal interface is used for scanning other parts of the patient. Abdominal, small organ, and peripheral vascular are some other examples.

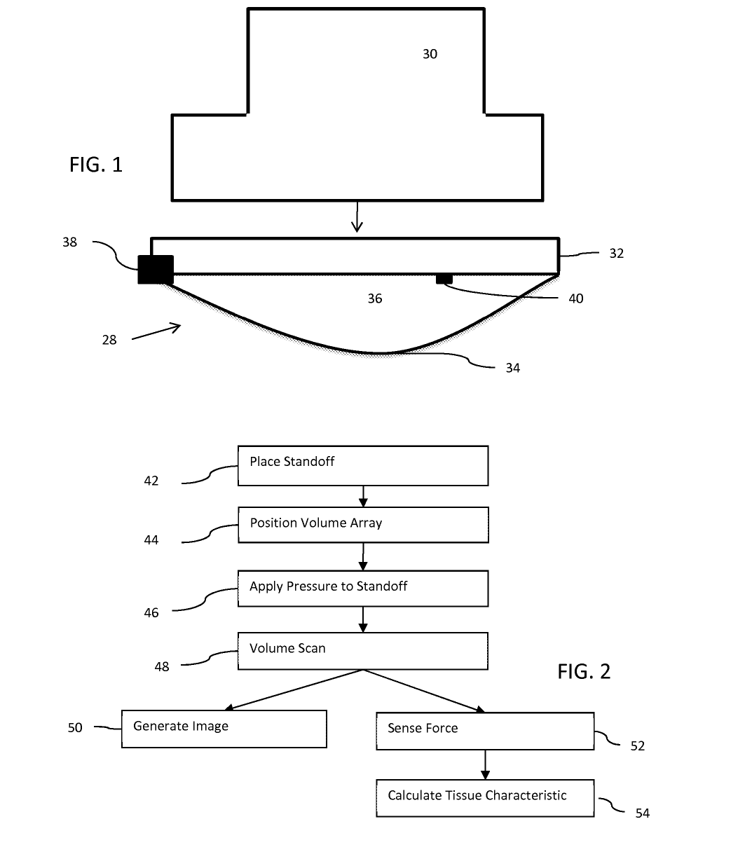

FIG. 1 shows one embodiment of a conformal interface for ultrasound imaging. The conformal interface is a standoff 28 for positioning between an ultrasound transducer 30 and the patient. The standoff 28 includes a transducer contact surface 32 connected with a patient conforming surface 34, forming a container filled with fluid 36. A port 38 for filling the container and a force sensor 40 are also shown. Additional, different, or fewer components may be provided, such as not having the port 38 and/or force sensor 40.

The transducer contact surface 32 is an elastomer, such as PEBAX. Other materials may be used. The material is sterilizable, so may be subjected to heat and steam for cleaning and re-use. The material is also acoustically conductive and provides a matched acoustic impedance to the fluid 36 and the transducer 30, such as having an acoustic impedance similar to water and tissue.

The transducer contact surface 32 is configured for acoustic contact with the ultrasound transducer 30. Any one or combination of various arrangements may be used to configure the transducer contact surface 32 for acoustic contact with the ultrasound transducer 30. To avoid artifacts, acoustic matching is provided. By minimizing acoustic mismatch in impedance, acoustic energy more likely travels through an interface or boundary. For scanning tissue, gel is used to provide contact free of air between the emitting face of the transducer 30 and the skin. The skin, gel, and patient have similar acoustic impedance, but air does not.

In one embodiment, the transducer contact surface 32 is configured for contact by being stiff or stiffer than the patient conforming surface 34. For example, the transducer contact surface 32 is thicker, such as being 3-4 times the thickness (e.g., 10-15 mils), of the patient conforming surface 34. Structure, such as ridges, may be used to stiffen the transducer contact surface 32. For example, a lip is formed around all or a part of the circumference of the transducer contact surface 32. The lip or ridges limit bending.

The transducer contact surface 32 is substantially flat and rigid. Substantially accounts for deviation from flat that is slight enough that contact between the rigid emitting face of the transducer 30 and the transducer contact surface 32 is maintained, at least through a gel, and without pockets of air. For example, the transducer contact surface 32 is sufficiently stiff that opposite edges do not deviate from planar by more than 0.5 cm while pressure is applied by the transducer 30 against the transducer contact surface 32 for ultrasound imaging. More or less deviation or amounts of bend may be provided. The stiffness allows the transducer 30 to be used on the transducer contact surface 32 for high-frequency (e.g., 2-10 MHz) scanning of the patient.

In another embodiment, the transducer contact surface 32 is configured for acoustic contact through shape. The emitting face of the transducer 30 is planar or curved and has a circumference of any shape. The transducer contact surface 32 has a mating shape and curvature. For example, the transducer 30 is 15 cm×15 cm. Likewise, the transducer contact surface 32 is 15 cm×15 cm or slightly (e.g., within 3 cm) larger. As another example, the transducer 30 has a diameter of at least 10 cm in any direction. The transducer contact surface 32 has a matching or slightly larger diameter in the directions.

In yet another embodiment, the transducer contact surface 32 is configured for acoustic contact through mating structures, such as alignment holes or rods. In one approach, a lip extends around a circumference of the transducer contact surface 32. The lip extends away from the transducer contact surface 32 and the patient, extending towards the transducer 30. The transducer 30 fits between the lip and against the transducer contact surface 32. For example, the square or rectangular emitting face of the transducer 30 fits within or surrounded by the lip where the lip defines a mating ellipsoid, circular, square or rectangular indention. Water, gel, or other acoustically matched mating material may be positioned within the indention for forming acoustic contact between the transducer 30 and the transducer contact surface 32.

The transducer contact surface 32 may be entirely flat or have bumps or other structure. The bumps or other structure may reduce friction or may be designed to increase friction. In one embodiment, gel is avoided during use. Instead, an adhesive lubricant is coated, deposited, imbedded, or formed on an etched transducer contact surface 32. The adhesive lubricant adheres to the transducer contact surface 32 and allows acoustic contact with the transducer 30. By pre-applying the adhesive lubricant, such as at manufacture, a permanent or semi-permanent (e.g., 3 or more uses) acoustic matching material is provided so that the transducer 30 or part of the transducer 30 may slide during scanning.

The patient contact surface 34 is a sheet of elastomer, such as PEBAX. Other materials may be used. The patient contact surface 34 is made from biocompatible, sterilizable, thin, good touch-feel material. Due to contacting skin, patient comfort is provided. Any thickness may be used while maintaining some flexibility. The patient contact surface 34 is thick enough to avoid wrinkling. In one example, the patient contact surface 34 is formed of material that is 1-5 mils thick. Greater or lesser thickness may be used.

The patient contact surface 34 is of the same or different material as the transducer contact surface 32. In one embodiment, different types of the same material are used. For example, the patient contact surface 34 is made from PEBAX SA MED, and the transducer contact surface 32 is made from lower friction coefficient PEBAX 7033.

The patient contact surface 34 is conformal to the patient. The patient has a generally flat or curved surface. For example, the breast is generally curved with a protrusion formed by the nipple. The patient contact surface 34 is a sheet of material that conforms to most or the entire surface of the patient when pressed against the patient. In one embodiment, the patient contact surface is elastic, stretching due to pressure. The flexible and elastic patient contact surface 34 takes on the shape of the patient over at least part of the surface 34.

The patient contact surface 34 has any shape at rest or while under pressure from the fluid 36 but not pressed against the patient. In the example of FIG. 1, the patient contact surface 34 is formed from a sheet of material connected to the transducer contact surface 32. The sheet may be flat other than caused by the fluid 36 or may have a bowl shape or curvature as represented in FIG. 1. Any depth may be provided, such as 2-3 inches at the center. In other embodiments, the patient contact surface 34 has other shapes, such as having an inverse bowl shape forming a cup for pre-disposed mating with a breast of a supine patient. In other embodiments, side walls are formed from the transducer contact surface 32 so that the patient contact surface 34 forms a drum membrane type surface.

The patient contact surface 34 is flat or smooth. Texture may be provided. In one embodiment, a pre-formed or permanent indentation is provided for mating with a nipple.

The patient contact surface 34 connects directly to the transducer contact surface 32. Adhesive, sonic welding, heat welding, or other connection may be used. In alternative embodiments, side walls or other intervening structure is provided between the patient and transducer contact surfaces 32, 34.

The connection forms a sealed container. The fluid 36 is maintained in the sealed container without leaking out. The seal is fixed or releasable. In one embodiment, the port 38 is provided. The port 38 is a one way valve or a two-way valve. The port 38 allows the addition and/or removal of fluid 36 from the sealed container. A cylinder or pump may be provided for hydraulic addition or removal of the fluid 36. In one embodiment, the port 38 connects through tubing to a syringe housing fluid 36 for injecting or removing some of the fluid 36.

The fluid 36 is viscous. For example, the fluid 36 is more viscous than water. Any fluid may be used, such as oil (e.g., cooking oil). The fluid 36 has a similar acoustic impedance as water and tissue, avoiding substantial mismatch with the patient and the sealed container (e.g., housing formed by the patient contact and transducer contact surfaces 32, 34).

The fluid 36 is housed in the sealed container under pressure. For example, the pressure is greater than 1 atmosphere. The port 38 or other structure used to inject during manufacture keeps the fluid 36 at the desired pressure. With the port 38, the pressure may be increased or decreased as needed. The pressure is settable by injection or removal.

By being pressure filled, fluid is more likely left between the patient and transducer 30 even under significant chestward compression by the operator. The fluid 36 ensures a uniform pressure field across the entire breast. The uniform pressure may provide a reduction in shadowing from connective tissue over the entire breast. The thick, viscous fluid enables the positioning of the fluid-filled bag onto the patient with pressure distributed over the entire contact surface. Less viscous fluids, such as water, may not allow for significant pressure to be applied. Water may result in less even pressure distribution by allowing contact between the transducer and patient contact surfaces 32, 34 without intervening fluid. The viscous fluid may slow down displacement and flow under operator chestward pressure.

The standoff 28 provides a viscous fluid contained in a housing. The housing is, in a first part, a flexible, elastic material and is, in a second part, a relatively more stiff material. The stiff part is non-deforming or deforms little as pressure is applied by the transducer, maintaining acoustic contact. The flexible, elastic part enables distributed pressure across all or most of the breast without patient discomfort due to a peak in pressure at a smaller region. The viscous fluid ensures a more uniform pressure across the breast and allows positioning for scanning. By being acoustically conductive, the standoff 28 may be used for ultrasound scanning. By being sterilizable, the standoff 28 may be used multiple times (e.g., three or more). The combination of viscous fluid and soft touch material allows for a significant chestward pressure to be applied to the entire breast, reducing shadowing with less patient discomfort. The standoff 28 is an economical, semi-permanent coupling device.

In one embodiment, one or more force sensors 40 are provided on or in the standoff 28. Any force sensor 40 may be used, such as a strain gauge, ultrasound for distance measuring, or pressure sensor. For example, a pressure sensor is inside the standoff 28. The pressure sensor in the sealed container measures the fluid pressure. The fluid pressure is responsive to the pressure of the fluid 36 within the standoff 28 and any chestward pressure applied against the standoff 28 by the transducer 30. Measuring the uniform pressure field may assist in sophisticated elasticity calculations since the force applied across the breast may be measured.

FIG. 2 is a flow chart diagram of one embodiment of a method for scanning a breast in medical diagnostic ultrasound volume imaging. The standoff 28 of FIG. 1 or a different standoff is used with an ultrasound imaging system to scan the breast. The scanning is of the entire breast or a portion of the breast without moving the standoff 28 relative to the patient's breast and/or without repositioning the transducer housing. Repositioning or moving may be provided to scan different parts of the patient.

The method is performed in the order shown or another order. For example, act 46 is performed while also performing act 48 and/or act 50. Additional, different, or fewer acts may be provided. For example, acts 50, 52, and/or 54 are not performed. As another example, acts for elasticity imaging or scanning configuration are performed.

In act 42, a bag filled with a viscous fluid under pressure is placed against the breast of the patient. For example, a sealed bag with the internal fluid pressure being greater than 1 atmosphere and with the viscous fluid comprising oil or other fluid more viscous than water is placed. The standoff of FIG. 1 may be applied. Gel or other acoustic coupling may be applied to the bag or the breast prior to placement.

In act 44, a volume scanning transducer is positioned against the bag. The transducer is positioned against the side of the bag opposite the breast. Gel or other acoustic coupling may be applied to the transducer or the bag prior to positioning. In one embodiment, the bag is infused or pre-coated with a semi-permanent lubricant for acoustic coupling and/or being able to move an array of the transducer with less friction along the bag.

The volume scanning transducer is a two-dimensional array, wobbler, or other transducer for scanning more than one plane with ultrasound while the transducer housing is stationary. In one embodiment, the transducer is a one-dimensional array mounted to a track. Gears or a pulley mechanically translate the one-dimensional array for volume scanning. Where the bag has a frame or other structure for accepting the transducer housing, a window or other cover may not be provided over the emitting face. In alternative embodiments, a cover is provided and the cover makes acoustic contact with the bag.

In act 46, pressure is applied to the breast. The transducer is pressed against the bag. The pressure is greater than resulting from gravity on the bag and the transducer. The sonographer's hand or a robotic arm applies the pressure. Any amount of pressure may be applied. The pressure applied to the bag is also applied to the patient.

As a result of the pressure, the flexible, fluid filled portion of the bag conforms to the breast of the patient. The breast is pushed to be flatter. The bag flexes and/or expands to conform to the shape of the breast. In this arrangement, the more rigid or flat portion of the bag acoustically contacts the transducer, and the flexible portion conforms to the breast. The flexible material and pressurized viscous fluid conform to the breast such that air gaps on the order of ½ wavelength or greater are prevented or not present. Similarly, the thickness, elasticity, and/or pressure make it more likely that the flexible material of the bag does not wrinkle.

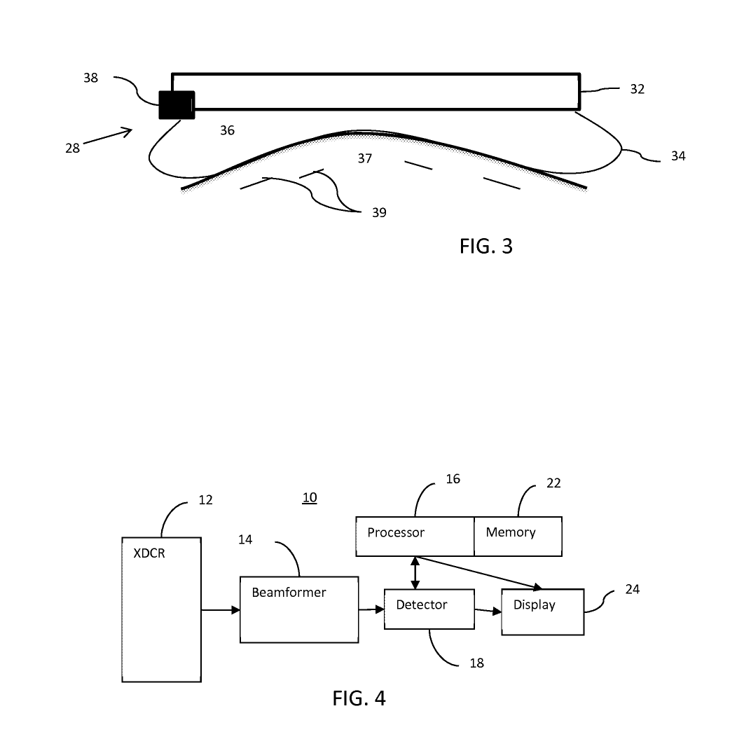

FIG. 3 shows an example. The patient contact surface 34 is pressed to the sides but some fluid 36 is maintained along the entire patient contact surface 34, causing a generally equal pressure to be applied along most or the entire breast 37. Rather than having a profile of pressure as a function of location that is peaked or parabolic (more pressure at the center and less pressure at the sides), the profile is more flat (more equalized pressure along a greater spatial extent).

In act 48 of FIG. 2, the breast is volume scanned. The scanning occurs through the bag. The volume scanning transducer array transmits acoustic energy focused within the breast. The acoustic energy passes through the bag and into the patient. At least 70%, 80%, 90% or more of the acoustic energy passes into the patient rather than being reflected by boundaries of the bag. Acoustic echoes responsive to the transmitted acoustic energy travel through the bag and arrive at the transducer.

The volume scanning transmits and receives along scan lines distributed throughout a volume (e.g., the breast). For example, multiple planes in the breast are scanned. The volume scan is performed with electronic steering. Alternatively, mechanical steering is provided along one direction and electronic in another, such as with a wobbler array. In one embodiment, a one-dimensional array is translated along a surface of the bag to scan different planes, forming the volume scan.

Due to the acoustic contact along the bag by the transducer array, all or much of the breast volume may be scanned with the bag and transducer housing in one position. For small to medium breasts, a single placement and positioning is used rather than separately scanning 2 or more (e.g., typical 4-5) volumes associated with different placement of the volume array relative to the breast. In alternative embodiments or for larger breasts, the bag may be replaced to another location on the breast for separately volume scanning different parts of the breast volume.

In act 50, an image is generated from the scanning. The scan generates electrical signals that are beamformed. The beamformed samples represent different voxels or locations in a three-dimensional grid or sampling pattern within the breast. Using an ultrasound system, the beamformed samples are detected, such as intensities for B-mode imaging or velocity, power, or variance estimates for color flow imaging. Filtering, scan conversion, interpolation to a three-dimensional Cartesian coordinate grid, or other processing is performed.

The image is generated from the detected data. A three-dimensional rendering may be created, such as rendering a two-dimensional image for display from the voxels distributed in three dimensions. Projection or surface rendering may be used. Alternatively or additionally, a planar image is generated. A plane through the volume is defined and the voxel data along the plane is used to generate a two-dimensional view of that plane through the breast. Multi-planar reconstruction may be provided.

Due to the pressure applied by the transducer to the bag, shadows are less likely in the image. Connective tissue is denser, so more greatly attenuates acoustic energy. As a result, returns or echoes from beyond the connective tissue relative to the transducer are weaker, causing shadowing. Having greater depth extent of the connective tissue causes more shadowing. By applying more equalized pressure, the connective tissue 39 (see FIG. 3 where lines represent the connective tissue) is forced into a more flat or horizontal distribution. This results in less shadowing.

In elasticity or strain imaging, a force is applied to the tissue. For example, the pressure from the transducer is applied. The strain, flexibility or elasticity of the tissue is then measured. The elasticity or strain may be measured without knowing how much force is applied. To determine characteristics of the tissue, such as the Young's modulus, the amount of force applied is used. In act 52, the force is sensed from within the bag. A pressure or other force sensor determines the amount of force applied to the tissue or skin. The forces applied within the breast may be extrapolated from the pressure in the bag or force applied to the skin. In act 54, a tissue characteristic is calculated using the data from the scanning (e.g., elasticity or strain data) and the force. Young's modulus or other tissue characteristics are calculated.

FIG. 4 shows a system 10 for medical diagnostic ultrasound imaging. The system 10 may be used with a standoff, such as the standoff 28 of FIG. 1, for scanning a patient, such as volume scanning a breast. The system 10 includes a transducer probe 12, a beamformer 14, a processor 16, a detector 18, a memory 22, and a display 24. Additional, different, or fewer components may be provided. For example, the system 10 includes a user interface. In one embodiment, the system 10 is a medical diagnostic ultrasound imaging system. In other embodiments, the processor 16 and/or memory 22 are part of a workstation or computer different or separate from an ultrasound imaging system. The workstation is adjacent to or remote from the ultrasound imaging system. In some embodiments, the transducer probe 12 is provided without other components.

In one embodiment, the system represents an automated breast volume scanner. The transducer probe 12 is provided for scanning the breast. The transducer probe 12 is handheld or may be part of an automated scanning system. For example, the transducer probe 12 is supported by a robotic arm or a support arm. Gravity, servos, motors, springs, hydraulics or other mechanism hold the transducer probe 12 in place against a patient's breast. Other applications than breast imaging may be provided.

The transducer probe 12 is a transducer array for medical diagnostic ultrasound imaging. The transducer probe 12 includes a probe housing and a transducer array. Additional, different, or fewer components may be provided, such as a cable and/or electronics.

The transducer probe 12 includes a planar array, a curved array, a two-dimensional array, a radial array, an annular array, or other multidimensional array of transducer elements. For example, the transducer probe 12 includes a multi- or two-dimensional array. Two-dimensional array has elements spaced in multiple directions (e.g., N×M where both N and M are greater than 1) but does not necessarily have an equal extent in each direction. Multi-dimensional arrays include 1.25D, 1.5D, 1.75D, annular, radial, or other arrangements of elements over an area rather than a line.

In an alternative embodiment, the transducer probe 12 has a one-dimensional array that connects with a guide. The guide is a rail, a pulley, a hydraulic system, a screw drive, mechanical linkage, ball bearings, rack and pinion, or other mechanism for guiding the transducer array in rotational or lateral movement. For example, the guide includes two grooves where the transducer array rests in the grooves and is connected to a pulley or chain. The grooves support the array to move generally perpendicular, such as in an elevation direction. A motor connects with the array, such as through a pulley or gears. The motor applies force to move the transducer array. Any speed of motion may be provided to translate or move the transducer array. The scan head is mechanically translated in the direction parallel to the short axis, causing the transmit plane to sweep across an entire volume. A controller operates the motor at the desired times and/or speed. Any type of motor may be used, such as a stepper motor, electric motor, or pump.

The transducer probe 12 includes a probe housing. For the breast imager, the probe housing is a pod or outer shell of plastic, fiberglass, metal, and/or other material. An acoustic window, such as the flexible bag with or without gel or other ultrasound transmissive substance between the transducer array and the pad, is provided. For example, the pad conforms to the shape of a compressed breast. Gel between the pad and the transducer array allows the adaptation and provides an acoustic path from the transducer array to the breast. Alternatively, the probe housing is part of a mammogram system or any other breast compression or scanning system.

In alternative embodiments for use scanning the breast or for other uses, the probe housing is for handheld use. The shape and surface texture of the probe housing includes a grip or handle for manual movement of the probe housing. An acoustic window, such as plastic or lens, may be provided.

The probe housing encases, surrounds most of, or is a protective frame work around the transducer array. The probe housing may include handles, grips, latches, connections, a transducer cable, or other components. Electronics may be provided within the probe housing, but the probe housing may be free of active (e.g., transistors, switches, or preamplifiers) electronics.

The acoustic elements of the transducer probe 12 are lead zirconate titanate (PZT) piezoelectric transduction material, ferroelectric relaxor or PVDF materials, capacitive membrane ultrasonic transducer (cMUT) materials, micro-machined membranes or beams, microelectromechanical devices, other piezoelectric material, or other means for acoustic-to-electric and/or electric-to-acoustic transduction. For example, the acoustic elements are cMUT or micromachined structures, such as at least one flexible membrane suspended over a gap with electrodes on each side of the gap for transducing between acoustic and electrical energies. Each acoustic element is formed from one or more, such as 4-8, tens or other numbers of membranes and gaps (i.e., “drums” or cMUT cells). The electrodes of each of the membranes and gaps for a given element are connected in common to form the single acoustic element.

All of the acoustic elements comprise a same type of material, but multiple types of acoustic transducer materials may be used for different acoustic elements. The acoustic elements have one of various possible shapes, such as triangular, rectangular, square, polygonal, hexagonal, circular, irregular, or any combination of shapes on the face of the acoustic element (i.e., portion of the element placed adjacent a volume to be scanned).

The transducer probe 12 converts between electrical signals and acoustic energy for scanning a region of the patient's body. The region of the body scanned is a function of the type of transducer array and position of the transducer probe 12 relative to the patient. A linear aperture may scan a rectangular or square, planar region of the body. As another example, a curved linear aperture may scan a pie shaped region of the body. Scans conforming to other geometrical regions or shapes within the body may be used, such as Vector™ scans. The scans are of a two-dimensional plane, such as scanning at different azimuth angles relative to the aperture. Different planes or different segments of a plane may be scanned by moving the transducer array. To scan a breast volume, the transducer array is also or instead moved mechanically to scan different elevation spaced planes.

The beamformer 14 is configured by hardware and/or software. For example, focus tables are used to determine the delays or phases for steering acoustic beams. Pursuant to software control, the desired waveforms are generated for transmit operation, and the desired receive process is implemented.

In one embodiment, the beamformer 14 includes transmitters or waveform generators for generating electrical waveforms for each element of a transmit aperture. The waveforms are associated with phase and amplitude. The waveforms for a given transmit event may have the same or different phasing. The electrical waveforms are relatively weighted and delayed to form an acoustic beam with a desired phase and amplitude characteristic. For example, the transmit beamformer includes amplifiers, phase rotators, and/or controllers to generate sequential, steered pulses with the desired phase and amplitude in relation to other acoustic beams. Converging, diverging or planar beams may be used.

The beamformer 14 may include receive beamformers, such as delays, phase rotators, amplifiers, and/or adders for relatively delaying and summing received signals to form one or more receive beams with dynamic focusing. For example, using shared processing, separate processing, or combinations thereof, a plurality (e.g., tens or hundreds) of parallel receive beamformers are provided to form a respective plurality of receive beams in response to a given transmit beam. Alternatively, the beamformer 14 includes a processor for Fourier or other analysis of received signals to generate samples representing different spatial locations of the scanned region. In other embodiments, only one or a few (e.g., nine or fewer) receive beams are generated for each transmit beam.

The receive beamformer connects with the receive elements of the transducer array after pre-amplification, any signal conditioning (e.g., filtering) and analog-to-digital conversion. The receive beamformer may be on-chip with the elements.

The transducer probe 12 and beamformer 14 are connected together, such as the transmit beamformer channels connecting through coaxial cables to the transducer probe 12. The transducer probe 12 and beamformer 14 are configured to scan a planar region or a segment of a planar region. The beamformer 14 is controlled or programmed to perform the scan. The beamformer parameters, such as relative delays and/or phasing for focus, apodization, beam amplitude, beam phase, frequency, or others, are set. The aperture for transmit and the aperture for receive on the transducer probe 12 is set. The beamformer 14 and transducer probe 12 are used to generate the waveforms for the aperture and convert the waveforms to acoustic energy for transmitting the beam. The beamformer 14 and transducer probe 12 are used to receive acoustic energy at the receive aperture, convert the acoustic energy to electrical energy, and beamform the received electrical signals.

Electric steering may be used to scan a plane. A volume scan may be performed using mechanical movement of the transducer array or further electric steering. Any pattern or distribution of scan lines and/or apertures may be used. Acoustic energy is transmitted in any of various now known or later developed scan patterns along each scan plane for acquiring data. The scan plane is then altered to another location in the volume by moving the transducer array. By moving the transducer array along the guide, a volume may be scanned. The volume is represented by data for a plurality of planes.

For each plane position, the beamformer is configured to scan the plane once. Alternatively, the plane is scanned multiple times but with different scan line angles in azimuth for compounding spatially. Different aperture locations may be used for scanning a given location from different angles.

For a given volume, the scans may be repeated. By repeating the scans, a sequence of frames of voxel data is obtained. Each frame represents the entire three-dimensional scanned volume, but may only represent smaller regions within the volume, such as a plane. By repeating the scanning, a plurality of frames of beamformed data representing the volume and/or plane is acquired. Any of scan line, part of frame, frame, or group of frame interleaving may be used.

The detector 18 is configured to detect data output by the beamformer 14 and responsive to the transducer array. The detector 18 is an ultrasound detector. The detector is configured by hardware and/or software to detect from the beamformed and/or interpolated data. Any detection may be used, such as B-mode, Doppler or color flow mode, harmonic mode, or other now known or later developed modes. B-mode and some harmonic modes use single pulse scan techniques for detection. The intensity of the received signals in the frequency band of interest is calculated. Multiple pulse techniques, such as flow mode estimation of velocity or energy, may be used.

The detector 18 detects the response to the transmit beams for the scan of the volume. The spatial and/or temporal resolution of the detected data is based on the beamforming or scanning resolution. Detected data representing the volume is provided.

The processor 16 is a rendering processor configured by hardware and/or software. The processor 16 is a general processor, control processor, application-specific integrated circuit, field-programmable gate array, graphics processing unit, digital circuit, analog circuit, digital signal processor, combinations thereof, or other now known or later developed device for generating a three-dimensional rendering of a volume scanned with different planes. The processor 16 is a single device or group of devices. For example, the processor 16 includes separate processors operating in parallel or sequence. As another example, the processor 16 includes a network of devices for distributed processing in parallel or sequence. In one embodiment, the processor 16 is a specific device for three-dimensional image rendering, such as a graphics processing unit, graphics card, or other device for rendering.

The processor 16 uses surface rendering, projection rendering, alpha blending, texturing, or other now known or later developed rendering. The data may be resampled to a regular voxel grid. Alternatively, the rendering is performed from data in a scan format, such as associated with the actual scan lines and/or interpolated scan lines. In yet other embodiments, the processor 16 is not provided or is a scan converter for generating a two-dimensional image representing a scanned plane or a reconstruction of a plane from a scanned volume.

The processor 16, the detector 18, or a separate processor generates images from the volume scan and/or plane scan or other data output from the detector 18. For example, grayscale and/or color coding is used to generate a B-mode, Doppler mode, or B-mode Doppler mode combination. Any image, such as a three-dimensional rendering, is output to the display 24.

The display 24 is a CRT, LCD, plasma, projector, printer, or other now known or later display device. The display 24 receives the image data from the processor 16 or other component and generates the image. A three-dimensional rendering, two-dimensional image, or other image is displayed.

The memory 22 is a tangible (non-transitory) computer readable storage medium, such as a cache, buffer, register, RAM, removable media, hard drive, optical storage device, or other computer readable storage media. The memory 22 is tangible by not being a signal, but a device. Computer readable storage media include various types of volatile and nonvolatile storage media. The memory 22 is accessible by the processor 16.

The memory 22 stores data representing instructions executable by the programmed processor 16, processor of the beamformer 14, and/or processor for scanning with ultrasound and/or controlling the motor of the transducer probe 12. The instructions for implementing the processes, methods and/or techniques discussed herein are provided on computer-readable storage media or memories. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

While the invention has been described above by reference to various embodiments, it should be understood that many changes and modifications can be made without departing from the scope of the invention. The above embodiments are examples. It is therefore intended that the foregoing detailed description be understood as an illustration of the presently preferred embodiments of the invention, and not as a definition of the invention. It is only the following claims, including all equivalents, that are intended to define the scope of this invention.

What is claimed is:

1. A conformal interface for ultrasound imaging, the conformal interface comprising:
a sealed container having a first surface stiffer than an opposing second surface, the second surface being conformal to an outside surface of a patient, and the first surface configured for acoustic contact with an ultrasound transducer array; and
a fluid more viscous than water in the sealed container.

2. The conformal interface of claim 1, wherein the first surface comprises a square or rectangular frame with a lip around a circumference of the frame and extending away from the second surface.

3. The conformal interface of claim 1, wherein the first surface is thicker than the second surface.

4. The conformal interface of claim 1, wherein the second surface is elastic.

5. The conformal interface of claim 1, wherein the second surface is PEBAX and the first surface is PEBAX.

6. The conformal interface of claim 5, wherein the first and second surfaces are different types of PEBAX.

7. The conformal interface of claim 1, wherein the first surface has a diameter of at least 10 cm in any direction and wherein the first surface is stiff such that opposite edges deviate from planar by no more than 0.5 cm while under hand applied pressure for ultrasound imaging.

8. The conformal interface of claim 1, wherein the first surface is substantially flat.

9. The conformal interface of claim 1, wherein the first surface comprises an adhesive lubricant.

10. The conformal interface of claim 1, wherein the fluid comprises an oil.

11. The conformal interface of claim 1, wherein the fluid is within the sealed container under a pressure greater than 1 atmosphere.

12. The conformal interface of claim 1, where the sealed container comprises a port configured to injection of the fluid and holding the fluid at a pressure greater than 1 atmosphere, the pressure settable by the injection.

13. The conformal interface of claim 1, further comprising a force sensor in the sealed container.

14. A method for scanning a breast in medical diagnostic ultrasound volume imaging, the method comprising:
placing a bag filled with a viscous fluid under pressure against the breast of the patient;
positioning a volume scanning transducer against the bag;
applying pressure with the volume scanning transducer to the bag, the pressure greater than caused by gravity; and
volume scanning the breast through the bag with the volume scanning transducer.

15. The method of claim 14 wherein the bag comprises a substantially rigid transducer surface and a flexible breast contact surface, wherein applying the pressure comprises conforming the flexible breast contact surface to the breast while the breast is subject to the pressure.

16. The method of claim 14, wherein placing comprises placing with the bag being a sealed bag with the pressure being greater than 1 atmosphere and with the viscous fluid being oil.

17. The method of claim 14, wherein volume scanning comprises scanning a plurality of planes through the breast, and further comprising generating an image from the scanning.

18. The method of claim 14, wherein applying the pressure comprises pressing with a sonographer's hand or a robotic arm, the bag conforming to an emitting face of the volume scanning transducer and conforming to the breast such that air gaps and wrinkles in the bag between the volume scanning transducer and the breast are not present.

19. The method of claim 14, further comprising:
sensing pressure within the bag; and
calculating a tissue characteristic as a function of the force and elasticity or strain data responsive to the scanning.

20. A standoff for ultrasound imaging, the standoff comprising:
a viscous fluid contained in a housing, the housing being, in a first part, a flexible, elastic material and being, in a second part, a relatively more stiff material, the viscous fluid and the housing being acoustically conductive.
